(19) United States
(12) Patent Application Publication
(10) Pub. No.: US 2015/0265614 A1
(43) Pub. Date: Sep. 24, 2015

(54) ORAL PREPARATION AND METHOD FOR PRODUCING THE SAME

(71) Applicant: CHUNG-ANG UNIVERSITY INDUSTRY-ACADEMY COOPERATION FOUNDATION, Seoul (KR)

(72) Inventors: Jong Hwi Lee, Seoul (KR); Hye Seung Lee, Seoul (KR); Min Kyung Lee, Suwon-si Gyeonggi-do (KR); Harim Bae, Incheon (KR); You Jin Kim, Gyeonggi-do (KR); Su Yeong An, Seoul (KR); Jong Seok Yeom, Seoul (KR); Dong Ryoul Shin, Seoul (KR); Seung Hwan Jung, Seoul (KR); Hyun Joo Song, Seoul (KR)

(21) Appl. No.: 14/429,373

(22) PCT Filed: Sep. 20, 2012
(86) PCT No.: PCT/KR2012/007514

§ 371 Date: Mar. 19, 2015

(30) Foreign Application Priority Data
Sep. 19, 2012 (KR) 10-2012-0103922

(51) Int. Cl. A61K 31/4985; A61K 9/16; A61K 9/00

(52) U.S. Cl. CPC A61K 31/4985; A61K 9/0056; A61K 9/1623; A61K 9/1682

(57) ABSTRACT

An oral preparation including: a porous disintegrative template; and an active ingredient supported in the pores of the disintegrative template, is excellent in physical/chemical stability, processibility, and fast-acting property as compared to the existing dosage form, and may block unpleasantness due to a bitter taste during the internal use and meet the medicine-taking compliance.

### CROSS-REFERENCE TO RELATED APPLICATIONS

This application claims priority under 35 USC §119(a) of a Korean Patent Application No. 10-2012-0103922 filed on Sep. 19, 2012, the subject matter of which is hereby incorporated by reference.

### BACKGROUND

1. Field of the Invention

The present invention relates to an oral preparation including a porous template in which an active ingredient is supported in the pores, and a method for producing the same.

2. Background Art

Recently, the ratio of the elderly population in the society has increased due to the extension of human life, but these elderly people are in deteriorated health states such as vision, hearing, memory, and physical ability as well as pharmacokinetic changes, and thus need an appropriate drug therapy. In particular, these people have difficulties in taking typical tablets or capsules, and alternative preparations for oral administration agents are required for elderly people from this viewpoint.

Disintegrating preparations, which are easily disintegrated or dissolved in the mouth, are very useful preparations not only for elderly people having difficulties in taking the existing tablets or capsules, but also for children, disabled people, patients in bed, and the busy moderns. Liquid prescription drugs are available as an alternative for tablets or capsules, but the liquid preparations are disadvantageous in low stability and inaccuracy in dose. In particular, when a drug is absorbed in the oral mucosa, the hepatic first pass may also be avoided, so that among drugs to be absorbed from the digestive tract, a fast releasing film may also applied to drugs which are susceptible to hepatic metabolism. However, since the drug in the preparations which are easily dissolved in the mouth is absorbed through the oral mucosa, the preparations have a problem in that a bitter taste or unpleasant taste is caused when the drug is absorbed.

### SUMMARY OF THE INVENTION

### Technical Problem

An object of the present invention is to provide an oral preparation containing an active ingredient and a method for producing the same.

### Technical Solution

As an example for solving the object, the present invention provides an oral preparation including: a porous disintegrative template; and an active ingredient supported in the pores of the porous template.

As an example, the porous template may include water-soluble sugars, and may be utilized in various ways for use such as, for example, a therapeutic agent for erectile dysfunction, or an anti-inflammatory analgesic drug.

As another example of the present invention, the oral preparation may be produced by a method including: preparing a porous template by freeze-drying a solution of water-soluble sugars; supplying an active ingredient solution to the prepared porous template; and drying the porous template to which the active ingredient solution is supplied.

### Effect of the Invention

The oral preparation of the present invention is excellent in physical/chemical stability, processibility, and fast-acting property as compared to the existing dosage form, and may block unpleasantness due to a bitter taste during the internal use and meet medicine-taking compliance.

### BRIEF DESCRIPTION OF DRAWINGS

FIG. 1 is a schematic view illustrating a cross-section of an oral preparation formulated into a film form according to an exemplary embodiment of the present invention.

FIG. 2 is a photograph illustrating a result in which the porous template prepared in Example 2 is observed by an electron microscope.

FIG. 3 is a photograph illustrating a result in which crystalline particulates of an active ingredient formed in the pores of the porous template prepared in Example 2 are observed by an electron microscope.

FIG. 4 is a photograph illustrating a result in which tadalafil crystalline particulates coated with ethyl cellulose formed in the pores of the porous template prepared in Example 3 are observed by an electron microscope.

FIG. 5 is a photograph illustrating a result in which the porous template prepared in Example 5 is observed by an electron microscope.

FIG. 6 illustrates a photograph in which crystalline particulates of an active ingredient formed in the pores of the porous template prepared in Example 5 are observed by an electron microscope.

FIG. 7 is a photograph illustrating a result in which crystals of a tadalafil raw material prepared in the Comparative Examples are observed by an optical microscope.

FIG. 8 is a graph illustrating a powder X-ray diffraction pattern of the tadalafil raw material.

FIG. 9 is a graph illustrating a powder X-ray diffraction pattern of the oral preparation prepared according to Example 2.

FIG. 10 is a graph illustrating a powder X-ray diffraction pattern of the oral preparation prepared according to Example 3.

FIG. 11 is a graph illustrating a powder X-ray diffraction pattern of the oral preparation prepared according to Example 4.

FIG. 12 is a graph comparing tadalafil release characteristics of the oral preparation prepared according to Example 2 with release characteristics of tadalafil crystals prepared according to the Comparative Examples.

FIG. 13 is a graph comparing tadalafil release characteristics of the oral preparation prepared according to Example 3 with release characteristics of tadalafil crystals prepared according to the Comparative Examples.

FIG. 14 is a graph comparing the initial tadalafil release characteristics of the oral preparation prepared according to Example 3 with the initial release characteristics of tadalafil crystals prepared according to the Comparative Examples.

FIG. 15 is a graph comparing tadalafil release characteristics of the oral preparation prepared according to Example 4 with release characteristics of tadalafil crystals prepared according to the Comparative Examples.

FIGS. 16, 17 and 18 are graphs illustrating a result in which the sizes of naproxen crystal particles prepared according to Examples 6 and 7 and Comparative Example 2, respectively, are measured.

### DETAILED DESCRIPTION OF THE INVENTION

The present invention relates to an oral preparation including: a porous disintegrative template; and an active ingredient supported in the pores of the porous template.

As an example, the porous template may be a porous template in which micropores are formed. The pores formed in the porous template may have an average diameter of 100 μm or less, or 20 μm or less. When the diameters of the pores are extremely large, the diameter may become larger than a desired size during the process in which a pharmacologically active ingredient in the pores is crystallized. The lower limit of the diameter of the pores is not particularly limited, and a smaller diameter is preferred because the smaller the diameter is, the smaller the size of the crystal particulates of the active ingredient crystallized in the micropores becomes, and in the present invention, the lower limit may be 0.5 μm or more, 1 μm or more, or 2 μm or more.

The porous template may include water-soluble sugars. The porous template according to the present invention includes water-soluble sugars, and thus is disintegrative, more specifically, has a fast disintegration property.

Water-soluble sugars may serve as an important ingredient which induces the sweet taste in the mouth and affects the sense of touch and the fast disintegration property. Specific kinds of water-soluble sugars are not particularly limited, and water-soluble sugars may be used without limitation as long as the sweet taste and water-soluble property are excellent. Examples of the water-soluble sugars include one or more selected from the group consisting of lactose, glucose, sucrose, fructose, levulose, maltodextrin, palatinose, mannitol, sorbitol, xylitol, and erythritol.

The porous template of the present invention includes water-soluble sugars, and thus may block the bitter taste of a pharmacologically active ingredient through the sweet taste of water-soluble sugars, and be easily dissolved in the mouth, if necessary.

The porous template may be composed only of water-soluble sugars. In some cases, the porous template may further include one or more additives selected from the group consisting of polyvinyl alcohol, polyethylene glycol, and polyacrylic acid in addition to water-soluble sugars. Through this, the physical strength of the porous template may be reinforced, and a storage property may be enhanced.

In the present invention, the active ingredient may be present in a crystal particulate form in the pores of the porous template, and may be present in an aggregate form in some cases. The shape of the crystallized particulate of the active ingredient is not particularly limited, and may be appropriately selected according to the raw material for the active ingredient. The oral preparation of the present invention includes an active ingredient in a crystal particulate form, and thus shows the crystal size much smaller than, for example, the case in which the existing pharmacologically active ingredient is crystallized, and may enhance physical/chemical stability, processibility, and fast-acting property.

In the present invention, the crystal size of the crystal particulate of the active ingredient is not particularly limited, and may be, for example, 50 nm to 100 μm, 50 nm to 10 μm, 100 nm to 10 μm, 1 μm to 10 μm, or 5 μm to 10 μm. By adjusting the crystal size to the range, water-solubility of the active ingredient may be prevented from being decreased.

As an example, the active ingredient may be in a state in which the active ingredient is coated with a water-soluble polymer. For example, the water-soluble polymer may be adsorbed on the surface of the crystal particulate of the active ingredient, thereby forming a coating layer. Through this, the crystal particulate of the active ingredient may be stabilized, and the active ingredient may be suppressed from being initially released, thereby blocking the bitter taste.

A specific kind of the water-soluble polymer is not particularly limited, but examples of the water-soluble polymer include one or more selected from the group consisting of alkyl cellulose, hydroxyalkyl cellulose, hydroxyalkyl alkylcellulose, carboxyalkyl cellulose, carboxyalkyl alkylcellulose, alkali metal salts of carboxyalkyl cellulose, carboxyalkyl cellulose ester, polyvinyl alcohol, polyvinyl pyrrolidone, polyalkylene glycol, polyalkylene oxide, carageenic acid, alginic acid, alkali metal of alginic acid, water-soluble chitosan, glucosan, polyaniline, cellulose acetate, polypyrrole, poloxamer, pluronic F-127 and phenylalanine-containing protein, lecithin, and carbopol.

In the present invention, examples of alkyl cellulose include methyl cellulose or ethyl cellulose, and the like; and examples of hydroxyalkyl cellulose include hydroxymethyl cellulose, hydroxyethyl cellulose, hydroxypropyl cellulose, or hydroxybutyl cellulose, and the like. Examples of hydroxyalkyl alkyl cellulose include hydroxyethyl methyl cellulose or hydroxypropyl methylcellulose, and the like; examples of carboxyalkyl cellulose include carboxymethyl cellulose, and the like; and examples of carboxyalkyl alkyl cellulose include carboxymethyl ethyl cellulose, and the like. Further, examples of alkali metal salts of carboxyalkyl cellulose include sodium carboxymethyl cellulose, and the like; examples of polyalkylene glycol include polyethylene glycol or polypropylene glycol, and the like; examples of polyalkylene oxide include polyethylene oxide, a copolymer of polypropylene oxide or ethylene oxide and propylene oxide, and the like, but are not limited thereto.

The oral preparation of the present invention may be formulated in various forms, and includes all of the cases in which a person skilled in the art easily modifies or supplements the oral preparation of the present invention. For example, as the oral preparation, examples of the oral preparation according to the present invention may be a dosage form of one or more selected from the group consisting of tablets, pills, hard and soft capsules, powders, powdered drugs, granules, pellets, and film agents. In addition, the oral preparation may be a single dosage form, and may be a complex dosage form in which two or more dosage forms are mixed in some cases.

These dosage forms may contain a surfactant, a diluent (for example: lactose, dextrose, sucrose, mannitol, sorbitol, cellulose, and glycine), and a lubricant (for example: silica, talc, stearic acid and magnesium or calcium salts thereof, and polyethylene glycol), in addition to the active ingredient. The tablets may also contain a binder such as magnesium aluminum silicate, starch paste, gelatin, tragacanth, methylcellulose, sodium carboxymethylcellulose and polyvinylpyrrolidone, and may contain a disintegrant such as starch, agar, alginic acid or a sodium salt thereof, and a pharmaceutical additive such as an absorbent, a colorant, a flavor, and a sweetener in some cases. The tablets may be produced by a typical mixing, granulating, or coating method.

As an example, the oral preparation may be a film dosage form. The film dosage form may have a thickness of 10 μm to 30,000 μm, 10 μm to 10,000 μm, 100 μm to 500 μm, 1,000 μm to 5,000 μm, or 100 μm to 200 μm. It is possible to form relatively uniform micropores by adjusting the thickness of the film dosage form within the range.

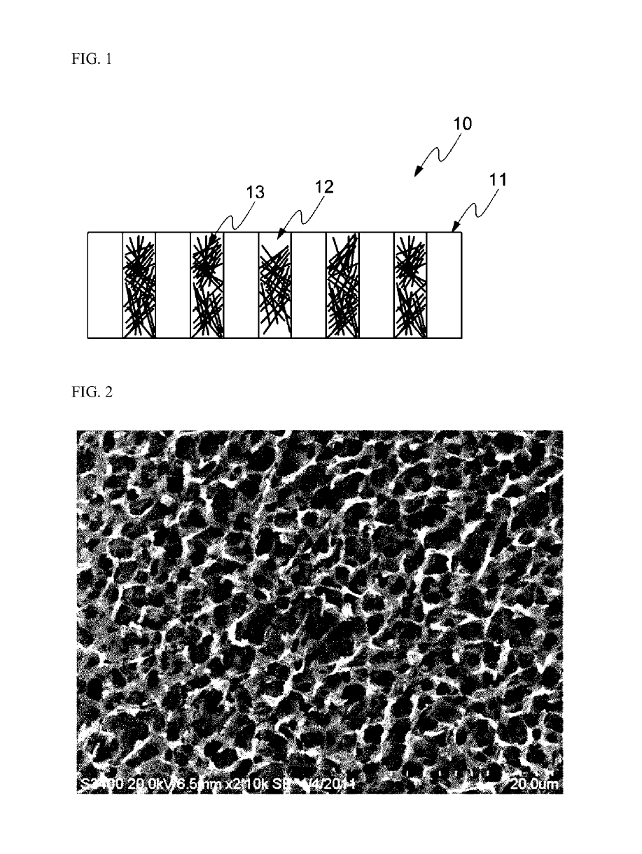

FIG. 1 is a view illustrating a film dosage form cross-sectional view of an oral preparation according to an exemplary embodiment of the present invention. As illustrated in FIG. 1, a film dosage form 10 of the present invention may have a structure including a porous template 11 having a micropore 12; and a crystal particulate 13 of an active ingredient supported in the pore 12 of the porous template 11. The present invention is an illustration of a film dosage form of FIG. 1 as an example, and may be formulated in various forms in addition to the film dosage form.

Furthermore, the pharmaceutically acceptable dose of the active ingredient, that is, the administration dose may vary depending on the age, gender, and weight of a subject to be treated, the specific disease or pathological state to be treated, the severity of the disease or pathological state, the administration route, and the prescriber's determination. The determination of the administration dose based on these factors is within the level of the person skilled in the art. A general administration dose may be 0.01 mg/kg/day to 1,000 mg/kg/day and 1 mg/kg/day to 40 mg/kg/day, but the administration dose is not intended to limit the scope of the present invention by any method.

The present invention proposes a novel dosage form which may support various active ingredients, and the kind of active ingredient to be supported is not particularly limited. The kind of active ingredient to be supported in the micropores of the porous template is not particularly limited, and the active ingredient may be used without limitation as long as the active ingredient is an active ingredient to be orally administered, but may be preferably an ingredient which may rapidly exhibit the effect through rapid dissolution. The active ingredient may be in the form of pharmacologically acceptable various active ingredients or salts thereof, and may be various pharmacological auxiliary ingredients which supplement or help the body's metabolism in some cases.

As an example, specific examples of the active ingredient according to the present invention include one or more selected from the group consisting of triclosan, cetyl pyridium chloride, domiphen bromide, quaternary ammonium salts, zinc compounds, sanguinarine, fluoride, alexidine, octonideine, EDTA, aspirin, acetaminophen, ibuprofen, ketoprofen, diflunisal, fenoprofen calcium, naproxen, tolmetin sodium, indomethacin, benzonatate, caramiphen, edisylate, menthol, dextromethorphan hydrobromide, chlophedianol hydrochloride, diphenhydramine, pseudoephedrine, phenylephrine, phenylprophanolamine, pseudoephedrine sulfate, bromophenyl amine maleate, chlorophenylamine maleate, carbinoxamine maleate, clemastine fumarate, dex-chlorpheniramine maleate, diphenhydramine hydrochloride, diphenhydramine citrate, diphenylpyraline hydrochloride, doxylamine succinate, promethazine hydrochloride, pyrilamine maleate, tripelennamine citrate, triprolidine hydrochloride, acrivastine, loratadine, brompheniramine, dexbrompheniramine, guaifenesin, ipecac, calcium iodide, terpin hydrate, loperamide, famotidine, ranitidine, omeprazole, lansoprazole, aliphatic alcohol, nicotine, caffeine, strychnine, picrotoxin, pentylenetetrazole, phenylhydantoin, phenobarbital, primidone, carbamazepine, ethosuximide, methosuximide, pensuccinimide, trimethadione, diazepam, benzodiazepine, phenacemide, pheneturide, acetazolamide, sulthiame, bromide, levodopa, amantadine, morphine, heroin, hydromorphone, metopon, oxymorphone, levorphanol, codeine, hydrocodone, xycodone, nalorphine, naloxone, naltrexone, salicylate, phenyl butazone, indomethacin, phenacetin, chlorpromazine, methotrimeprazine, haloperidol, clozapine, reserpine, imipramine, tranylcypromine, phenelzine, lithium, apomorphine, sildenafil, tadalafil, vardenafil, ondansetron, donepezil, zolpidem tartrate, granisetron, montelukast, Pholcodine, butyl scopolamine, fentanyl citrate, oxycodone hydrochloride, buprenorphine hydrochloride, escitalopram oxalate, rivastigmine tartrate, esomeprazole magnesium, aripiprazole, zolmitriptan, rizatriptan benzoate, telmisartan, risperidone, benzocaine, cetirizine hydrochloride, bambuterol hydrochloride, galantamine hydrobromide, lercanidipine hydrochloride, paroxetine hydrochloride, meloxicam, tolterodine tartrate, doxazosin mesylate, and pharmacologically acceptable salts thereof.

The active ingredient may be a pharmacologically active ingredient, and examples of the oral preparation include one or more selected from the group consisting of: therapeutic agents for diabetes mellitus, such as glimepiride and pioglitazone; therapeutic agents for insomnia, such as zolpidem and eszopiclone; therapeutic agents for genitourinary diseases, such as tolterodine and trospium; therapeutic agents for obesity, such as sibutramine; enzymatic agents such as streptokinase; therapeutic agents for gastric ulcer, such as omeprazole; antitussives and apophlegmatics, such as theophylline and clenbuterol; therapeutic agents for skin diseases, such as finasteride; antiemetics, such as ondansetron; antidepressants, such as fluoxetine; antihistamines, such as fexofenadine hydrochloride; antipyretics, analgesics and antiphlogistics, such as aspirin, ibuprofen, ketoprofen, and meloxicam; hormone drugs such as testosterone; therapeutic agents for circulatory diseases, such as fleodipine, atorvastatin, amlodipine camsylate, doxazosin, simvastatin, and lercanidipine; therapeutic agents for digestive system diseases, such as famotidine, ranitidine, and lansoprazole; therapeutic agents for cardiovascular diseases, such as amlodipine and nitroglycerin; therapeutic agents for psychoneurotic disorders, such as paroxetine; therapeutic agents for erectile dysfunction, such as sildenafil, tadalafil, and vardenafil; therapeutic agents for Alzheimer's disease, such as donepezil; therapeutic agents for osteoporosis; therapeutic agents for arthritis; therapeutic agents for epilepsy; muscle relaxants; cerebral function enhancers; therapeutic agents for schizophrenia; immunosuppressants; antibiotics, such as ampicillin and amoxicillin; anticancer agents; anticancer therapeutic supplements; vaccines; oral cleansers; antianemics; therapeutic agents for constipation; therapeutic agents for allergic diseases; anticoagulants; and all in one cold and flu capsules.

As an example, the oral preparation may be a therapeutic agent for erectile dysfunction, including a phosphodiesterase-5 (PDE-5) inhibitor as an active ingredient. The specific kind of the PDE-5 inhibitor is not particularly limited, but examples of the PDE-5 inhibitor include one or more selected from the group consisting of vardenafil, sildenafil, tadalafil, udenafil, mirodenafil, and pharmacologically acceptable salts thereof, and more preferably tadalafil, udenafil, mirodenafil, and pharmacologically acceptable salts thereof.

As another example, the oral preparation may be an anti-inflammatory analgesic drug including a nonsteroidal anti-inflammatory ingredient as an active ingredient. As the nonsteroidal anti-inflammatory ingredient, commercially available various ingredients may be used, and for example, naproxen ((+)-(s)-2-(6-methoxynaphthalen-2-yl)propanoic acid), and the like may be used.

Further, the active ingredient of the present invention may be an ingredient which aids or enhances activities in addition to the pharmacologically active ingredient. For example, the oral preparation of the present invention may be a health function food or a health supplement food. Specifically, the preparation may be one or more selected from the group consisting of vitamins, nutritional supplements, and lactobacillus preparations.

As an example, the oral preparation may contain other ingredients and the like which may impart synergistic effects to a main desired effect within a range not impairing the main effect. For example, in order to improve physical properties, the oral preparation may further include additives such as a perfume, a colorant, a pesticide, an antioxidant, a preservative, a humectant, a thickener, inorganic salts, an emulsifier, and a synthetic polymer material. In addition, the oral preparation may further include supplemental ingredients such as water-soluble vitamins, oil-soluble vitamins, a polymer peptide, a polymeric polysaccharide, and a seaweed extract. The ingredients may be suitably selected and blended by a person skilled in the art according to a dosage form or use purpose without difficulties, and the addition amount may be selected within a range not impairing the object and effect of the present invention.

In addition, the present invention provides a method for producing the oral preparation previously described.

As an example, the method for preparing the oral preparation may include:

preparing a porous template by freeze-drying a solution of water-soluble sugars;

supplying an active ingredient solution to the prepared porous template; and

drying the porous template to which the active ingredient solution is supplied.

The producing process for each step is specifically observed as follows.

First, in the preparing of a porous template, the solution of water-soluble sugars may include water-soluble sugars and water, and may further include one or more additives selected from the group consisting of polyvinyl alcohol, polyethylene glycol, and polyacrylic acid in some cases. That is, the solution of water-soluble sugars may be prepared by dissolving water-soluble sugars in water, or dissolving a mixture of water-soluble sugars and the additive in water. The solution of water-soluble sugars prepared by dissolving the mixture of water-soluble sugars and the additive in water may enhance physical strength of a porous template through the additive. The details on the water-soluble sugars are the same as those described above, and thus will be omitted.

As an example, the solution of water-soluble sugars may include 1 part by weight to 40 parts by weight, 5 parts by weight to 30 parts by weight, or 5 parts by weight to 20 parts by weight of water-soluble sugars based on 100 parts by weight of a solvent. The solvent is not particularly limited, and water may be used. By adjusting the content of the water-soluble sugars to the range, micropores in the porous template may be prevented from being non-uniformly formed.

As another example, the solution of water-soluble sugars may include 1 part by weight to 40 parts by weight or 5 parts by weight to 20 parts by weight of water-soluble sugars; and 0.1 part by weight to 10 parts by weight or 0.5 part by weight to 5 parts by weight of an additive based on 100 parts by weight of the solvent. The solvent is not particularly limited, and water may be used. By adjusting the content of the additive to the range, micropores in the porous template may be prevented from being non-uniformly formed.

The term “part by weight” used in the present invention means a weight ratio. Furthermore, the prepared solution of water-soluble sugars may be stored at a temperature of 20° C. to 70° C. or 30° C. to 60° C. for 6 to 24 hours, or 10 to 15 hours, such that the water-soluble sugars and/or the additive may be uniformly mixed.

And then, a porous template having micropores may be produced by freeze-drying the solution of water-soluble sugars. Specifically, the porous template may be produced by applying a solution of water-soluble sugars on a substrate to which a mold is attached, freezing the applied solution of water-soluble sugars by using a coolant, and subjecting the material perfectly solidified by the freezing to sublimation drying by a freeze dryer.

The specific kind of the substrate to which the mold is attached is not particularly limited, and for example, a glass substrate may be used in the present invention. The method of applying the solution of water-soluble sugars to the substrate to which the mold is attached is not also particularly limited, and any means typically used in the art may be adopted without limitation. Further, the kind of coolant used to freeze the solution of water-soluble sugars is not also particularly limited, and any material generally used in the art may be used without limitation. In the present invention, liquid nitrogen is used as an example of the coolant, but the example is not limited thereto.

The time for freeze-drying the solution of water-soluble sugars is not particularly limited, and may be continued until only the solid content of the water-soluble sugars; or the water-soluble sugars and the additive remains by subjecting the solidified material to sublimation drying, and may be, for example, 3 hours to 72 hours. When the freeze-drying time is extremely short, the solution is not perfectly freeze-dried, so that moisture remains, and the porous template may be dissolved by the remaining moisture.

Next, the method proceeds to the supplying of an active ingredient solution to a porous template prepared.

The active ingredient solution may be prepared by dissolving a pharmacologically active ingredient; or a pharmacologically active ingredient and a water-soluble polymer in one or more organic solvents selected from the group consisting of alcohol, alkyl acetate, dimethylformamide, dimethyl sulfoxide, acetone, anisole, acetic acid, butyl methyl ether, ethyl ether, ethyl formate, formic acid, pentane, heptane, methyl ethyl ketone, and methyl isobutyl ketone. That is, the active ingredient solution may be prepared by dissolving the active ingredient in an organic solvent, or dissolving a mixture of the active ingredient and the water-soluble polymer in an organic solvent. The details on the active ingredient and the water-soluble polymer are the same as those described above, and thus will be omitted.

For example, the active ingredient solution may include 1 part by weight to 40 parts by weight, 3 parts by weight to 30 parts by weight, or 5 parts by weight to 20 parts by weight of the active ingredient based on 100 parts by weight of the organic solvent. By adjusting the content of the active ingredient to the range, the effect may be exhibited within an effective range of the active ingredient, and the one-time dose may be satisfied.

Further, the active ingredient solution may further include 0.1 part by weight to 10 parts by weight, or 0.5 part by weight to 5 parts by weight of a water-soluble polymer based on 100 parts by weight of the organic solvent. By adjusting the content of the water-soluble polymer to the range, the pharmacologically active ingredient is perfectly coated with the water-soluble polymer, and furthermore, it is possible to prevent crystals from being formed only of a water-soluble polymer.

The active ingredient solution may be stored at room temperature for 10 minutes to 3 hours or 30 minutes to 2 hours, or subjected to ultrasonic wave treatment, such that the ingredients in the solution become uniform.

The prepared active ingredient solution is supplied to the porous template, and the specific method is not particularly limited. For example, the active ingredient solution may be supplied by a method of applying the solution on the porous template. When the active ingredient solution is applied on the porous template, the active ingredient solution may flow into the micropores of the porous template, and then be supported in the micropores. The method of applying the active ingredient solution on the porous template is not particularly limited, and for example, the active ingredient solution may be applied on the fast-porous template by using a dropper or pipette, but the method is not limited thereto.

The active ingredient solution is subjected to a step of supplying the active ingredient solution to the porous template, and then drying the active ingredient solution.

The oral preparation may be formulated in various forms during the process of drying the porous template. During the drying of the porous template, the active ingredient is crystallized while the organic solvent in the active ingredient solution is evaporated. Since crystallization of the active ingredient in the micropores of the porous template proceeds, the size of crystal particles of the active ingredient to be formed is very small, and a fast-acting property may be enhanced.

As an example, when the active ingredient solution includes the active ingredient, the aqueous polymer, and the organic solvent, the organic solvent is evaporated during the drying, and crystal particulates of the active ingredient coated with the water-soluble polymer may be formed while the water-soluble polymer is adsorbed on the surface of the crystal particles of the active ingredient.

The temperature at which the porous template is dried is not particularly limited, but may be in a range of, for example, 5° C. to 60° C., 20° C. to 50° C., 10° C. to 30° C., or 15° C. to 30° C. By adjusting the drying temperature to the range, crystallization of the active ingredient may be efficiently induced, and stability of the dosage form may be secured.

The method of drying the porous template is not also particularly limited, and for example, the porous template may be dried by using an oven, or naturally dried.

The time for drying the porous template is not particularly limited, and may be appropriately selected such that crystallization of the active ingredient may be sufficiently achieved, and for example, the porous template may be dried for 3 hours to 10 days, 3 hours to 5 days, 1 day to 5 days, or 3 hours to 7 days. By adjusting the time for drying to the range, the organic solvent in the active ingredient solution is completely dried, and the production efficiency may be prevented from being decreased.

### EXAMPLES

Hereinafter, the present invention may be specifically described through the Examples and the like, but the scope of the present invention is not limited thereby.

### Example 1

### Preparation of Solution of Water-Soluble Sugars

A solution of water-soluble sugars was prepared by putting 1 g of a water-soluble sugar mannitol into a 10 ml-glass test tube, and then adding 9 g of water to the glass test tube, and stirring the mixture.

### Preparation of Porous Template

0.05 ml of the prepared solution of water-soluble sugars was thinly applied on a glass substrate (25 mm×37 mm (width×length)) to which a mold with a size of 15 mm×25 mm (width×length) was attached. The applied solution of water-soluble sugars was frozen by liquid nitrogen, and a porous template was prepared by subjecting the sample perfectly solidified by freezing to sublimation drying in a freeze dryer (FD-1000 freeze dryer, (manufactured by) EYELA Co., pressure: 5.6 Pa, and temperature: −45° C.) for 24 hours. The pores formed in the porous template have an average diameter of 30 μm.

### Preparation of Active Ingredient Solution

A PDE-5 inhibitor solution was prepared by putting 0.5 g of a PDE-5 inhibitor tadalafil raw material (Sialis, (manufactured by) Glenmark Generics Ltd.) into a 5 ml-glass test tube, and then adding 4.5 g of an organic solvent dimethylformamide to the glass test tube, and stirring the mixture. Subsequently, the glass tube was stored at room temperature for 1 hour such that the prepared PDE-5 inhibitor solution became uniform.

### Application of Active Ingredient Solution on Porous Template

0.05 ml of the prepared PDE-5 inhibitor solution was uniformly applied on the porous template by using a pipette.

### Drying of Porous Template on which Active Ingredient was Applied

An oral preparation in a film dosage form was produced by naturally drying the porous template on which the PDE-5 inhibitor solution was applied at room temperature for 24 hours.

### Example 2

An oral preparation in a film dosage form was produced by performing the same manner as in Example 1, except that the solution of water-soluble sugars and the solution of a pharmacologically active ingredient were each prepared by the following methods.

### Preparation of Solution of Water-Soluble Sugars

A solution of water-soluble sugars was prepared by putting 0.9 g of a water-soluble sugar mannitol and 0.1 g of an additive polyvinyl alcohol into a 10 ml-glass test tube, and then adding 9 g of water to the glass test tube, and stirring the mixture. In order to allow the water-soluble sugars and the additive in the solution of water-soluble sugars to be uniformly mixed, the prepared solution of water-soluble sugars was stored at a temperature of 50° C. for 12 hours.

### Preparation of Active Ingredient Solution

A PDE-5 inhibitor solution was prepared by putting 0.5 g of a PDE-5 inhibitor tadalafil raw material (Sialis, (manufactured by) Glenmark Generics Ltd.) into a 5 ml-glass test tube, and then adding 4.5 g of an organic solvent dimethylformamide to the glass test tube, and subjecting the mixture to ultrasonic wave treatment.

### Example 3

An oral preparation in a film dosage form was produced by performing the same manner as in Example 1, except that the solution of water-soluble sugars and the solution of a pharmacologically active ingredient were each prepared by the following methods.

### Preparation of Solution of Water-Soluble Sugars

A solution of water-soluble sugars was prepared by putting 0.9 g of a water-soluble sugar mannitol and 0.1 g of an additive polyvinyl alcohol into a 10 ml-glass test tube, and then adding 9 g of water to the glass test tube, and stirring the mixture. In order to allow the water-soluble sugars and the additive in the solution of water-soluble sugars to be uniformly mixed, the prepared solution of water-soluble sugars was stored at a temperature of 50° C. for 12 hours.

### Preparation of Active Ingredient Solution

A PDE-5 inhibitor solution was prepared by putting 0.45 g of a PDE-5 inhibitor tadalafil raw material (Sialis, (manufactured by) Glenmark Generics Ltd.) and 0.05 g of a water-soluble polymer ethyl cellulose into a 5 ml-glass test tube, and then adding 4.5 g of an organic solvent dimethylformamide to the glass test tube, and stirring the mixture.

### Example 4

An oral preparation in a film dosage form was produced by performing the same manner as in Example 1, except that the solution of water-soluble sugars and the solution of a pharmacologically active ingredient were each prepared by the following methods.

### Preparation of Solution of Water-Soluble Sugars

A solution of water-soluble sugars was prepared by putting 0.9 g of a water-soluble sugar mannitol and 0.1 g of an additive polyvinyl alcohol into a 10 ml-glass test tube, and then adding 9 g of water to the glass test tube, and stirring the mixture. In order to allow the water-soluble sugars and the additive in the solution of water-soluble sugars to be uniformly mixed, the prepared solution of water-soluble sugars was stored at a temperature of 50° C. for 12 hours.

### Preparation of Active Ingredient Solution

A PDE-5 inhibitor solution was prepared by putting 0.45 g of a PDE-5 inhibitor tadalafil raw material (Sialis, (manufactured by) Glenmark Generics Ltd.) and 0.05 g of a water-soluble polymer pluronic F-127 ((manufactured by) BASF Co., Ltd.) into a 5 ml-glass test tube, and then adding 4.5 g of an organic solvent dimethylformamide to the glass test tube, and subjecting the mixture to ultrasonic wave treatment.

### Example 5

A mixture of drug particles in a particulate form and the water-soluble sugars was prepared by preparing a solution of water-soluble sugars, manufacturing a porous template by using the solution, and evaporating and crystallizing the drug in the pores.

### Preparation of Solution of Water-Soluble Sugars

A solution of water-soluble sugars was prepared by putting 0.9 g of a water-soluble sugar lactose and 0.1 g of an additive polyethylene glycol into a 10 ml-glass test tube, and then adding 2 g of water to the glass test tube, and stirring the mixture. In order to allow the water-soluble sugars and the additive in the solution of water-soluble sugars to be uniformly mixed, the prepared solution of water-soluble sugars was stored at room temperature for 2 hours.

### Preparation of Porous Template

A porous template was prepared by putting 0.4 ml of the prepared solution of water-soluble sugars into a glass petri dish (diameter 16 mm, height 2 mm), freezing the glass petri dish by liquid nitrogen, and subjecting the sample perfectly solidified by freezing to sublimation drying in a freeze dryer (FD-1000 freeze dryer, (manufactured by) EYELA Co., pressure: 5.6 Pa, and temperature: −45° C.) for 24 hours. The micropores of the porous template had an average diameter of 5 μm.

### Crystallization of Active Ingredient

A 6.7 wt % ethanol solution of a nonsteroidal anti-inflammatory drug (NSAID) agent naproxen ((+)-(s)-2-(6-methoxynaphthalen-2-yl)propanoic acid) was prepared in a 5 ml-glass test tube and stirred for 2 hours, and then 0.2 ml of the solution was applied to the porous water-soluble sugar template, and then evaporated at room temperature for 24 hours, and then crystals were obtained.

### Example 6

A mixture of drug particles in a particulate form and the water-soluble sugars was prepared by preparing a solution of water-soluble sugars, manufacturing a porous template by using the solution, and evaporating and crystallizing the drug in the pores.

### Preparation of Solution of Water-Soluble Sugars

A solution of water-soluble sugars was prepared by putting 2.7 g of a water-soluble sugar mannitol and 0.3 g of an additive polyvinyl alcohol (PVA) into a 20 ml-glass test tube, and then adding 7 g of water to the glass test tube, and stirring the mixture. In order to allow the water-soluble sugars and the additive in the solution of water-soluble sugars to be uniformly mixed, the prepared solution of water-soluble sugars was stored at a temperature of 60° C. for 12 hours.

### Preparation of Porous Template

A porous template was prepared by applying 0.4 ml of the prepared solution of water-soluble sugars on a silicon plate (diameter 16 mm, height 2 mm), freezing the silicon plate by liquid nitrogen, and subjecting the sample perfectly solidified by freezing to sublimation drying in a freeze dryer (FD-1000 freeze dryer, (manufactured by) EYELA Co., pressure: 5.6 Pa, and temperature: −45° C.) for 24 hours. The micropores of the porous template had an average diameter of 10 μm.

### Crystallization of Active Ingredient

A 10 wt % ethanol solution of a nonsteroidal anti-inflammatory drug (NSAID) agent naproxen ((+)-(s)-2-(6-methoxynaphthalen-2-yl)propanoic acid) was prepared in a 10 ml-glass test tube and stirred for 2 hours, and then 0.15 ml of the solution was applied to the porous water-soluble sugar template, and then evaporated at room temperature for 24 hours, and then crystals were obtained.

### Example 7

An oral preparation in a film dosage form was produced by performing the same manner as in Example 6, except that the solution of water-soluble sugars and the solution of a pharmacologically active ingredient were each prepared by the following methods.

### Preparation of Solution of Water-Soluble Sugars

A solution of water-soluble sugars was prepared by putting 3.15 g of a water-soluble sugar mannitol and 0.35 g of an additive polyvinyl alcohol (PVA) into a 20 ml-glass test tube, and then adding 6.5 g of water to the glass test tube, and stirring the mixture. In order to allow the water-soluble sugars and the additive in the solution of water-soluble sugars to be uniformly mixed, the prepared solution of water-soluble sugars was stored at a temperature of 60° C. for 12 hours.

### Preparation of Porous Template

A porous template was prepared by applying 0.4 ml of the prepared solution of water-soluble sugars on a silicon plate (diameter 16 mm, height 2 mm), freezing the silicon plate by liquid nitrogen, and subjecting the sample perfectly solidified by freezing to sublimation drying in a freeze dryer (FD-1000 freeze dryer, (manufactured by) EYELA Co., pressure: 5.6 Pa, and temperature: −45° C.) for 24 hours. The micropores of the porous template had an average diameter of 10 μm.

### Crystallization of Active Ingredient

A 10 wt % ethanol solution of a nonsteroidal anti-inflammatory drug (NSAID) agent naproxen ((+)-(s)-2-(6-methoxynaphthalen-2-yl)propanoic acid) was prepared in a 10 ml-glass test tube and stirred for 2 hours, and then 0.15 ml of the solution was applied to the porous water-soluble sugar template, and then evaporated at room temperature for 24 hours, and then crystals were obtained.

### Comparative Example 1

A PDE-5 inhibitor solution was prepared by putting 0.5 g of a PDE-5 inhibitor tadalafil raw material (Sialis, (manufactured by) Glenmark Generics Ltd.) into a 5 ml-glass test tube, and then adding 4.5 g of an organic solvent dimethylformamide to the glass test tube, and stirring the mixture. Subsequently, the glass tube was stored at room temperature for 1 hour such that the prepared PDE-5 inhibitor solution became uniform. And then, tadalafil crystals were obtained by thinly applying the prepared PDE-5 inhibitor solution on a glass substrate (25 mm×37 mm (width×length)) to which a mold with a size of 15 mm×25 mm (width×length) was attached, and evaporating the organic solvent through drying.

### Comparative Example 2

A solution for particle size analysis was prepared by putting 0.015 g of a nonsteroidal anti-inflammatory drug (NSAID) agent naproxen ((+)-(s)-2-(6-methoxynaphthalen-2-yl)propanoic acid) into a 5 ml-glass test tube, and then adding 3 ml of water in which 0.003 g of HPC had been dissolved thereto and dissolving the raw material.

### Experimental Example 1

The observation was made by using an electron microscope in order to confirm the structure of the porous templates and the crystal particles of the active ingredient, which were prepared in the Examples and the like, and the observation was made by using an optical microscope in order to see the crystal size of the tadalafil raw material prepared in the Comparative Examples.

FIG. 2 illustrates an electron microscope photograph of the porous template including mannitol and polyvinyl alcohol, which was prepared according to Example 2. As illustrated in FIG. 2, it can be confirmed that the porous template prepared in Example 2 had micropores with a diameter of 2 μm to 10 μm.

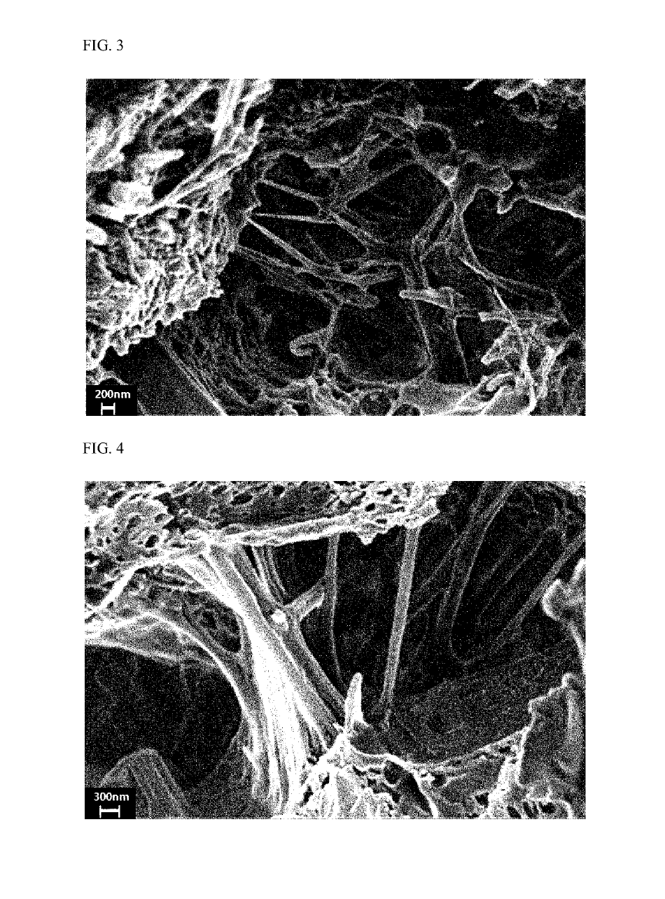

FIG. 3 illustrates an electron microscope photograph of tadalafil crystal particulates crystallized in the pores of the porous template prepared in Example 2. As illustrated in FIG. 3, it can be confirmed that the tadalafil crystal particulates included in the oral preparation in Example 2 had a needle-like shape and had a size of 100 nm to 10 μm.

FIG. 4 illustrates a result in which tadalafil crystalline particulates coated with ethyl cellulose formed through crystallization in the pores of the porous template according to Example 3 are observed by an electron microscope. As illustrated in FIG. 4, it can be confirmed that the complex crystalline particulates of ethyl cellulose and tadalafil included in the oral preparation prepared in Example 3 had a needle-like shape and had a size of 100 nm to 10 μm, were coated with ethyl cellulose, and thus became thicker than tadalafil crystalline particulates of the attached FIG. 3.

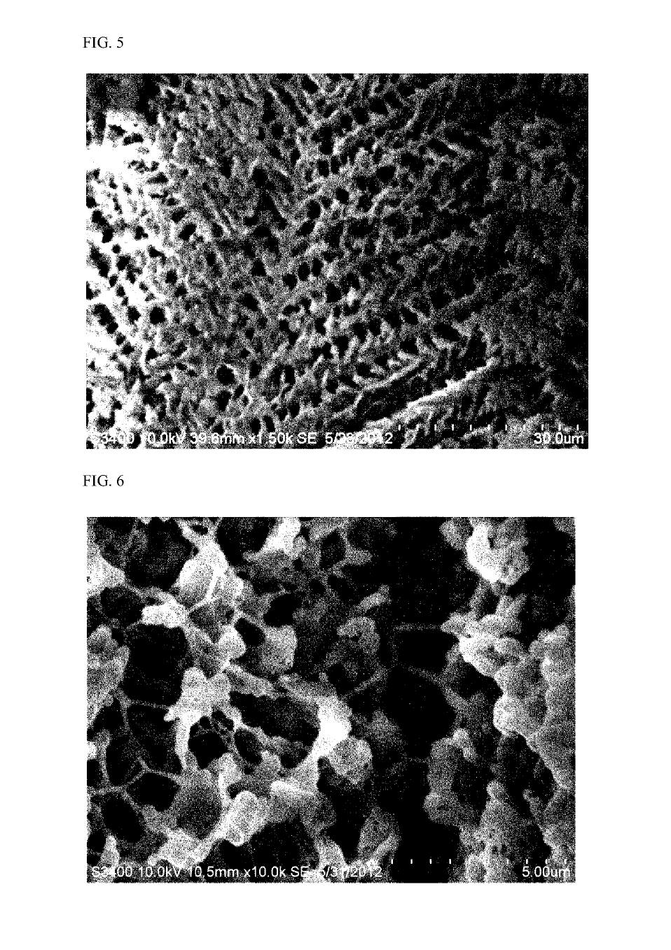

FIG. 5 illustrates a result in which the porous template prepared in Example 5 is observed by an electron microscope. As illustrated in FIG. 5, it can be confirmed that in the porous template prepared in Example 5, micropores with an average diameter of about 5 μm are formed.

FIG. 6 illustrates a result in which crystalline particulates of naproxen crystallized in the pores of the porous template prepared in Example 5 are observed by an electron microscope. As illustrated in FIG. 6, it can be confirmed that in the porous template prepared in Example 5, crystalline particulates of naproxen in a rod form were formed in the pores.

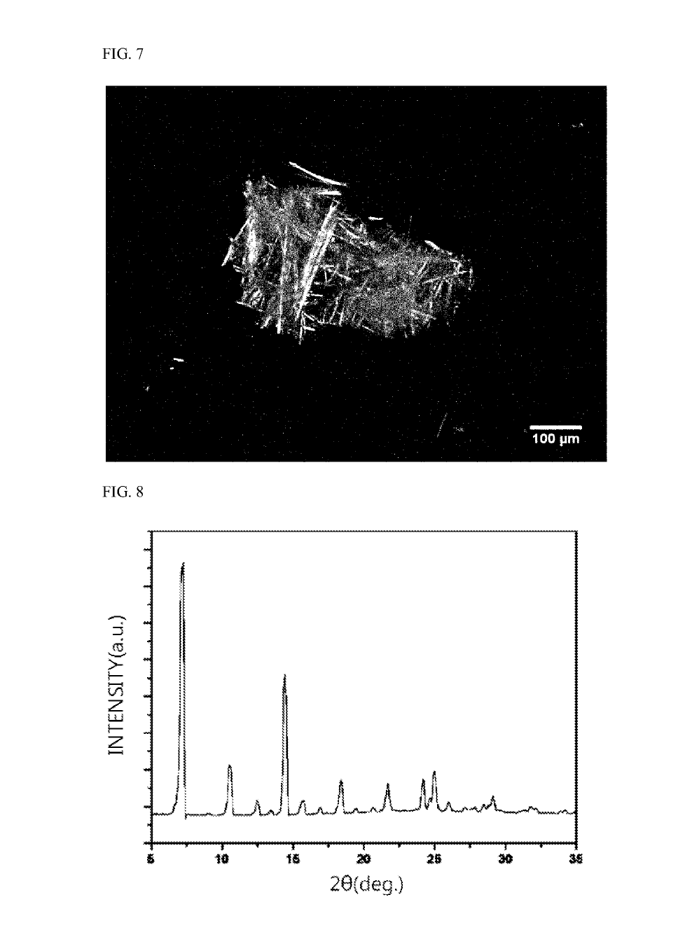

FIG. 7 illustrates a result in which crystals of the tadalafil raw material prepared in the Comparative Examples are observed by an optical microscope. As illustrated in FIG. 7, it can be confirmed that the crystal size of the tadalafil raw material is several hundred μm, and shows a significant difference with the size of the crystalline particulates of the tadalafil prepared in the Examples.

Therefore, the crystal size of the active ingredient included in the oral preparation according to the present invention is significantly smaller than the crystal of the crystals included in the existing dosage form, and through this, the surface area may be maximized to enhance the water-solubility and enhance the fast-acting property in the organism.

### Experimental Example 2

In order to see the complex crystal form of the oral preparation prepared in the Examples, a powder X-ray diffraction pattern was observed by using a powder X-ray diffraction analyzer (New D8-advance, (manufactured by) Bruker AXS Inc.).

FIG. 8 is a graph illustrating a powder X-ray diffraction pattern of a tadalafil raw material (Sialis, (manufactured by) Glenmark Generics Ltd.) according to the Comparative Examples.

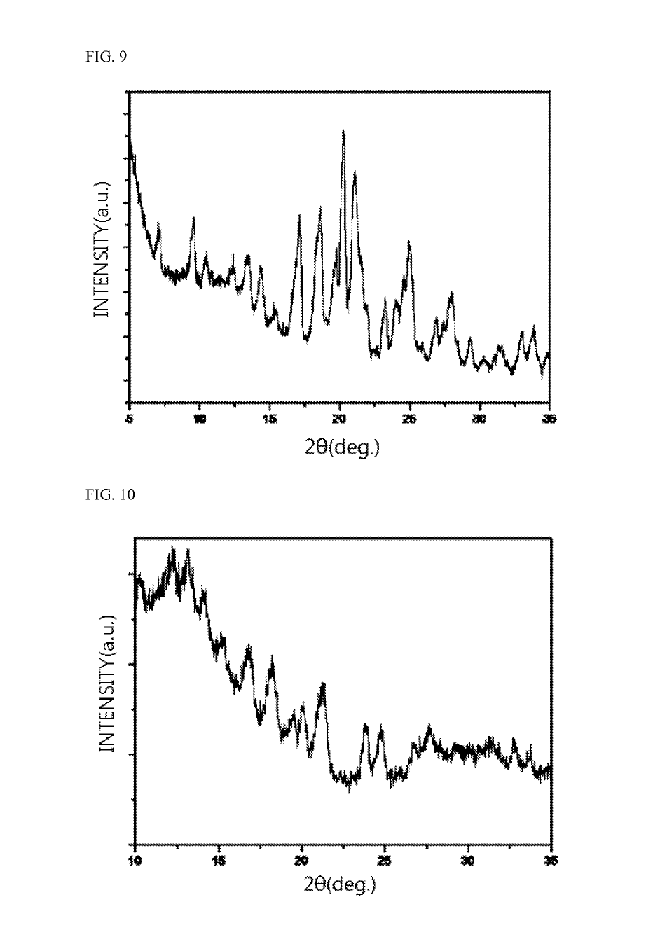

FIG. 9 is a graph illustrating a powder X-ray diffraction pattern of the oral preparation prepared according to Example 2. As illustrated in FIG. 9, it can be confirmed that the oral preparation prepared in Example 2 has a complex crystal form of mannitol, polyvinyl alcohol, and tadalafil obtained through crystallization of tadalafil in the micropores of the porous template including mannitol and polyvinyl alcohol.

FIG. 10 is a graph illustrating a powder X-ray diffraction pattern of the oral preparation prepared in Example 3. As illustrated in FIG. 10, it can be confirmed that the oral preparation prepared according to Example 3 has a complex crystal form of mannitol, polyvinyl alcohol, tadalafil, and ethyl cellulose obtained through crystallization of tadalafil and ethyl cellulose in the micropores of the porous template including mannitol and polyvinyl alcohol.

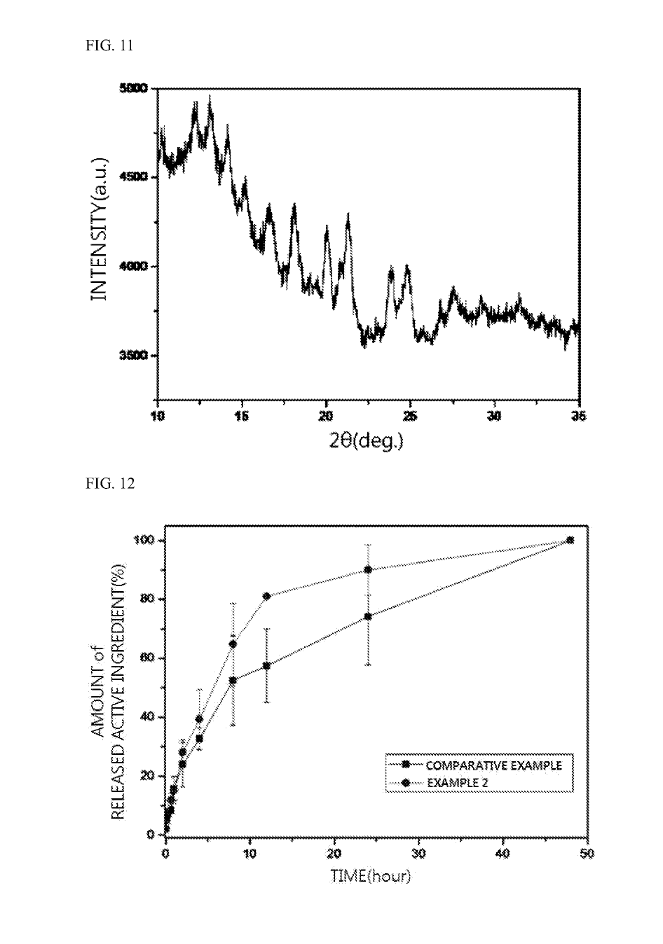

FIG. 11 is a graph illustrating a powder X-ray diffraction pattern of the oral preparation prepared in Example 4. As illustrated in FIG. 11, it can be confirmed that the oral preparation prepared in Example 4 has a complex crystal form of mannitol, polyvinyl alcohol, tadalafil, and pluronic F-127 obtained through crystallization of tadalafil and pluronic F-127 in the micropores of the porous template including mannitol and polyvinyl alcohol.

### Experimental Example 3

In order to see release characteristics of the oral preparation prepared in the Examples and the crystals of the tadalafil prepared in the Comparative Examples, the solubility of tadalafil was analyzed by the following method. Specifically, the oral preparation and the crystals of tadalafil prepared in Examples 2 to 4 and the Comparative Examples were each put into a 100 ml-beaker, and 100 ml of distilled water was added thereto at 36° C. to 38° C. Subsequently, a magnetic bar was put into the beaker, and the beaker was continuously agitated at 100 rpm while the release characteristic experiment was performed. Thereafter, a sample was collected from each beaker at each of 2, 10, 20, and 40 minutes and 1, 2, 4, 8, 12, 24, and 48 hours, a sample which had not been dissolved was filtered through a 0.2 μm-cellulose acetate syringe filter, and then the solubility of each of the tadalafil crystal particles was analyzed by using a high-performance liquid chromatography (HPLC). In this case, the quantification was made by comparing the concentration of mannitol and the absorption amount according to the kind of water-soluble polymer at 260 nm which is the maximum absorption wavelength of tadalafil.

FIG. 12 is a graph comparing tadalafil release characteristics of the oral preparation prepared in Example 2 with release characteristics of tadalafil crystals prepared in the Comparative Examples.

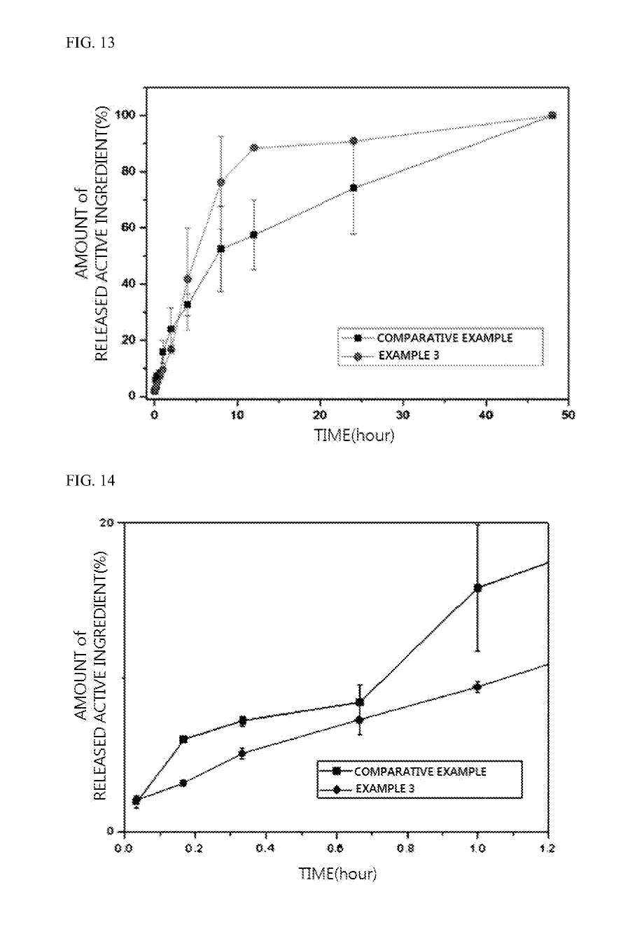

FIG. 13 is a graph comparing tadalafil release characteristics of the oral preparation prepared in Example 3 with release characteristics of tadalafil crystals prepared in the Comparative Examples.

FIG. 14 is a graph comparing the initial tadalafil release characteristics of the oral preparation prepared in Example 3 with the initial release characteristics of tadalafil crystals prepared in the Comparative Examples. As illustrated in FIG. 14, it can be seen that the oral preparation of the present invention including the crystal particulates coated with a water-soluble polymer has an effect of suppressing the initial release of tadalafil. Therefore, the oral preparation of the present invention may block the bitter taste of tadalafil through the effect of suppressing the initial release of tadalafil according to the coating of the water-soluble polymer.

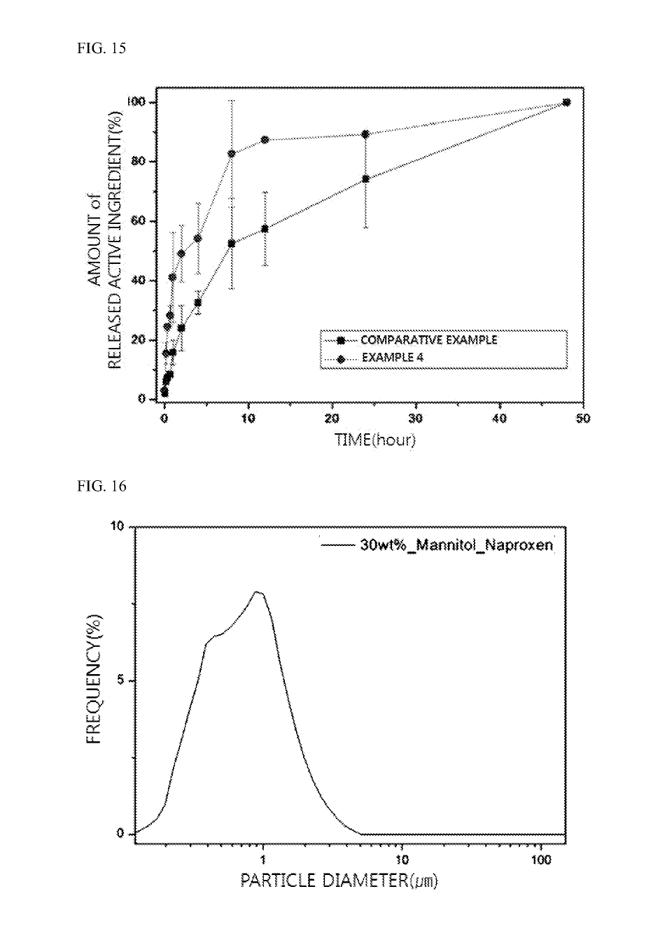

FIG. 15 is a graph comparing tadalafil release characteristics of the oral preparation prepared in Example 4 with release characteristics of tadalafil crystals prepared in the Comparative Examples.

As illustrated in FIGS. 12, 13, and 15, it was found that the oral fast-disintegrating film according to the present invention had released about 80% to about 90% of the crystal particles of tadalafil at the time point in which 12 hours had elapsed, but about 55% of the tadalafil crystals according to the Comparative Examples had been released. That is, the oral preparation according to the present invention includes crystalline particulates of tadalafil, may rapidly exhibit the efficacy through the rapid release because the crystal size becomes much smaller than that of the existing tadalafil crystal, or rapidly exhibit the efficacy through the rapid release after being absorbed in the body while the initial release may be suppressed to exhibit the effect of blocking the bitter taste in the mouth.

### Experimental Example 4

The sizes of the crystal particles of naproxen prepared in Examples 6 and 7 and Comparative Example 2 were observed. Specifically, the degree of dispersion of the particle sizes of a nonsteroidal anti-inflammatory drug (NSAID) agent naproxen ((+)-(S)-2-(6-methoxynaphthalen-2-yl)propanoic acid) was observed by using HORIBA LA-910 LASER SCATTERING PARTICLE SIZE ANALYZER. The observation results are each illustrated in FIGS. 16 to 18.

FIG. 16 illustrates the degree of dispersion of the particle sizes of particulates of naproxen prepared in Example 6. As illustrated in FIG. 16, it was found that the size of crystalline particulates of naproxen in the pores of the porous template prepared in Example 6 was 1 μm or less, and the average particle diameter was 0.835 μm.

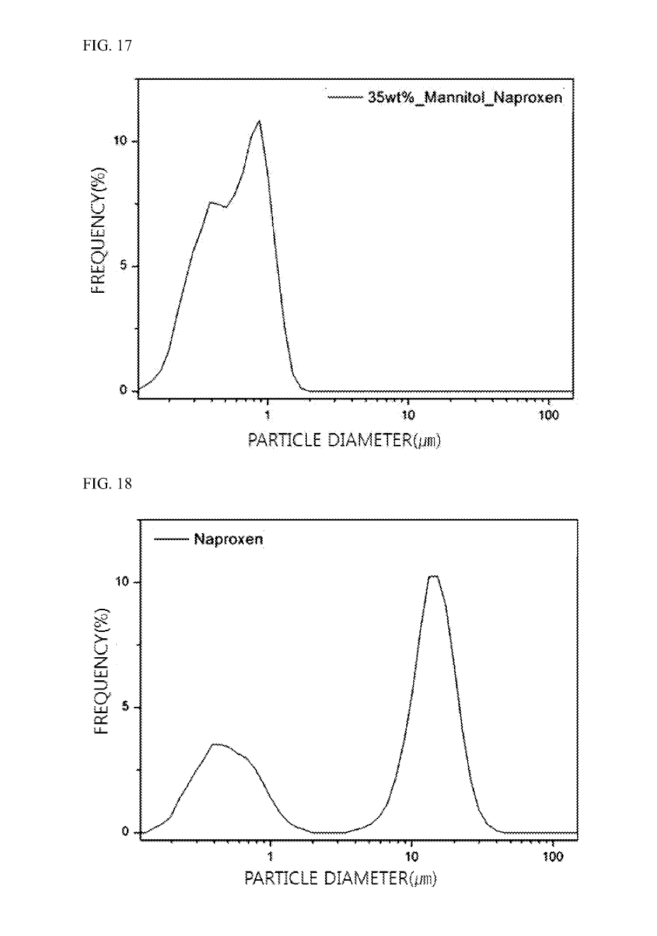

FIG. 17 illustrates the degree of dispersion of the particle sizes of particulates of naproxen prepared in Example 7. As illustrated in FIG. 17, it was found that the size of crystalline particulates of naproxen in the pores of the porous template prepared in Example 7 was 1 μm or less, and the average particle diameter was 0.616 μm. Further, in comparison with Example 6, it could be confirmed that the concentration of the aqueous solution of the porous template was so high that the pores became relatively small, and crystals of naproxen in a smaller particulate form could be obtained.

FIG. 18 illustrates the degree of dispersion of the particle sizes of the naproxen raw material prepared in Comparative Example 2. As illustrated in FIG. 18, it was found that the average crystal size of the naproxen raw material was 9.5 μm, showing a significant difference in comparison with the size of crystalline particulates of naproxen prepared in Examples 6 and 7.

### EXPLANATION OF CODES

10: Film dosage form
11: Porous template
12: Pore
13: Crystal particulates of an active ingredient

### INDUSTRIAL APPLICABILITY

The oral preparation according to the present invention may be utilized as various dosage forms for the fields such as pharmacy or health food.

What is claimed is:

1. An oral preparation comprising:
a porous disintegrative template; and
an active ingredient supported in the pores of the porous template.

2. The oral preparation of claim 1, wherein the pores formed in the porous template have an average diameter of 0.5 μm to 100 μm.

3. The oral preparation of claim 1, wherein the porous template comprises water-soluble sugars.

4. The oral preparation of claim 3, wherein the water-soluble sugars comprise one or more selected from the group consisting of lactose, glucose, sucrose, fructose, levulose, maltodextrin, palatinose, mannitol, sorbitol, xylitol, and erythritol.

5. The oral preparation of claim 1, wherein the active ingredient is present in a crystal particulate form.

6. The oral preparation of claim 5, wherein the active ingredient is present in a particulate form having an average diameter of 50 nm to 100 μm.

7. The oral preparation of claim 1, wherein the preparation is a dosage form of one or more selected from the group consisting of tablets, pills, hard and soft capsules, powders, powdered drugs, granules, pellets, and film agents.

8. The oral preparation of claim 1, wherein the active ingredient comprises a pharmacologically active ingredient, and
the oral preparation is one or more selected from the group consisting of therapeutic agents for diabetes mellitus, therapeutic agents for insomnia, therapeutic agents for genitourinary diseases, therapeutic agents for obesity, enzymatic agents, therapeutic agents for gastric ulcer, antitussives and apophlegmatics, therapeutic agents for skin diseases, antiemetics, antidepressants, antihistamines, antipyretics, analgesics and antiphlogistics, hormone drugs, therapeutic agents for circulatory diseases, therapeutic agents for digestive system diseases, therapeutic agents for cardiovascular diseases, therapeutic agents for psychoneurotic disorders, therapeutic agents for erectile dysfunction, therapeutic agents for osteoporosis, therapeutic agents for arthritis, therapeutic agents for epilepsy, muscle relaxants, cerebral function enhancers, therapeutic agents for schizophrenia, immunosuppressants, antibiotics, anticancer agents, anticancer therapeutic supplements, vaccines, oral cleansers, antianemics, therapeutic agents for constipation, and all in one cold and flu capsules.

9. The oral preparation of claim 8, wherein the preparation is a therapeutic agent for erectile dysfunction, which comprises a phosphodiesterase-5 (PDE-5) inhibitor as an active ingredient.

10. The oral preparation of claim 8, wherein the preparation is an anti-inflammatory analgesic drug comprising a nonsteroidal anti-inflammatory ingredient as an active ingredient.

11. The oral preparation of claim 1, wherein the preparation is a health functional food or a health supplement food.

12. A method for producing an oral preparation, the method comprising:
preparing a porous template by freeze-drying a solution of water-soluble sugars;
supplying an active ingredient solution to the prepared porous template; and
drying the porous template to which the active ingredient solution is supplied.

13. The method of claim 12, wherein the solution of water-soluble sugars comprises 1 part by weight to 40 parts by weight of water-soluble sugars based on 100 parts by weight of water.

14. The method of claim 12, wherein the active ingredient solution comprises 1 part by weight to 40 parts by weight of the active ingredient based on 100 parts by weight of the organic solvent.
